(19) United States
(12) Patent Application Publication
(10) Pub. No.: US 2026/0049360 A1
(43) Pub. Date: Feb. 19, 2026

(54) Therapeutic Strategies To Reduce Thrombotic Risk In Factor V Leiden (FVL) Carriers

(71) Applicant: Regeneron Pharmaceuticals, Inc., Tarrytown, NY (US)

(72) Inventors: Tanima De, Tarrytown, NY (US); Dan Chalothorn, Tarrytown, NY (US); KehDih Lai, Tarrytown, NY (US); Jonas Bovijn, Tarrytown, NY (US); Luca Andrea Lotta, Tarrytown, NY (US); Lori Morton, Tarrytown, NY (US)

(21) Appl. No.: 19/301,022

(22) Filed: Aug. 15, 2025

Related U.S. Application Data
(60) Provisional application No. 63/683,863, filed on Aug. 16, 2024.

(51) Int. Cl. C12Q 1/6886; C07K 16/36; C12N 15/113

(52) U.S. Cl. CPC C12Q 1/6886; C07K 16/36; C12N 15/113; C12N 2310/11; C12N 2310/14; C12Q 2600/118; C12Q 2600/156

(57) ABSTRACT

Methods of reducing the risk of venous thromboembolism (VTE) in a subject undergoing anticoagulant treatment by selectively depleting Factor V Leiden (FVL) in a subject that is heterozygous for FVL, or replacing FVL in the genome of a subject that is homozygous for FVL with a functional Factor V gene, and methods of identifying a subject undergoing anticoagulant treatment who is at risk of developing VTE are disclosed herein.

### FIELD

The present disclosure generally relates to methods of reducing the risk of venous thromboembolism (VTE) in a subject undergoing anticoagulant treatment by selectively depleting Factor V Leiden (FVL) in a subject that is heterozygous for FVL, or replacing FVL in the genome of a subject that is homozygous for FVL with a functioning Factor V gene, and to methods of identifying a subject undergoing anticoagulant treatment who is at risk of developing VTE.

### BACKGROUND

Factor V (FV or F5) synthesis principally occurs in the liver. Thrombin activates factor V, and once activated, it converts prothrombin to thrombin. Activated protein C (APC), an inhibitor of coagulation, degrades factor V. In the presence of thrombomodulin, thrombin acts to decrease clotting by activating protein C. Factor V Leiden (FVL; p.Arg506Gln) is a common missense variant in coagulation factor V (FV), a clotting factor in the blood. This missense can increase the chance of developing abnormal blood clots, most commonly in the legs or lungs. The replacement of arginine with glutamine at amino acid 506 abolishes the Arg506 cleavage site for APC in factor V. FVL increases the risk of thrombosis as APC cannot bind and inactivate factor V. Therefore, as factor V is not inactivated, it continues to be active and increases thrombosis risk.

FVL is common in Europeans (allele frequency combining all ancestries, 2.7%; 1 in 19 individuals carry the variant). Individuals heterozygous for FVL are at 2-fold higher risk of venous thromboembolism (VTE) (p<1×10⁻³⁰⁰), while homozygous FVL individuals have 6-fold higher risk (p=6.1×10⁻⁷⁸). Approximately 28% of FVL homozygotes have VTE. In people with a first VTE, each copy of FVL was associated with 50% higher risk of VTE recurrence. Although the variant was not associated with major bleeding risk (ORHETS: 0.93, p=0.06; ORHOM: 1.03, p=0.88), an increased bleeding risk (OR, ˜3) was observed when treated with anti-coagulants, approximately the same as non-carriers. In addition, FVL accounts for >95% of APC resistance. Standard anti-coagulant therapies, as well as factor XI and factor XII blockers, are currently used in FVL carriers.

### SUMMARY

The present disclosure provides methods of reducing the risk of VTE in a subject undergoing anticoagulant treatment, the methods comprising: determining or having determined whether the subject is heterozygous or homozygous for FVL by performing or having performed a sequence analysis on a biological sample obtained from the subject to determine if the subject has a genotype comprising FVL; and i) selectively depleting FVL in a subject that is heterozygous for FVL; or ii) replacing FVL in the genome of the subject that is homozygous for FVL with a functioning Factor V gene.

The present disclosure also provides methods of identifying a subject undergoing anticoagulant treatment who is at risk of developing VTE, the methods comprising: determining or having determined the presence or absence of an FVL nucleic acid molecule in a biological sample obtained from the subject; wherein: when the subject has a nucleic acid molecule encoding FVL, then the subject has an increased risk of developing VTE; and when the subject does not have a nucleic acid molecule encoding FVL, then the subject does not have an increased risk of developing VTE.

### BRIEF DESCRIPTION OF THE DRAWINGS

The accompanying figures, which are incorporated in and constitute a part of this specification, illustrate several features of the present disclosure and are intended to exemplify non-limiting embodiments of the present disclosure.

FIG. 1 shows FVL is a common variant associated with large effect increase in VTE risk.

FIG. 2 shows risk settings amplify absolute risk for VTE and FVL is a risk factor for excess risk in all those settings.

FIG. 3 shows anti-coagulant treatment is associated with higher risk for major bleeding across FVL genotypes.

FIG. 4 shows FVL carriers have similar risk of major bleeding as non-carriers while on anti-coagulant treatment.

FIG. 5 shows pseudo-homozygosity for FVL is associated with a biochemical phenotype as severe as homozygous FVL, despite reduced FV antigen levels.

FIG. 6 shows proportion with VTE is similar between pseudo-homozygous and homozygous FVL individuals.

FIG. 7 shows depletion of FVL combined with donor plasma containing normal FV ameliorates APC resistance.

FIG. 8 shows that in heterozygous-like dilutions, selective depletion of FVL ameliorates APC resistance.

FIG. 9 shows swapping of FVL A1-A2 domain with FV A1-A2 domain following cleavage by thrombin or Xa may ameliorate APC resistance.

FIG. 10 shows that plasma from an FVL homozygous individual diluted to 1% with FV-immunodepleted plasma leads to reduced coagulation potential, but does not ameliorate APC resistance.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horses, cows, and pigs), companion animals (such as, for example, dogs and cats), laboratory animals (such as, for example, mice, rats, and rabbits), and non-human primates. In some embodiments, the subject is a human. In some embodiments, the human is a patient under the care of a physician.

It has been observed in accordance with the present disclosure that for FVL homozygotes, plasma samples from Amish FVL carriers showed that in vitro depletion of FVL down to 1% does not ameliorate APC resistance and thrombin generation is substantially reduced but FV depletion to that level is expected to result in significant major bleeding risk. Replacing FVL with normal FV protein corrects APC resistance in vitro. Also, FVL pseudo-homozygosity (FVL heterozygotes with pLOF in opposite haplotype; a natural model for partial knockdown of FVL) has the same VTE risk as FVL homozygotes. Therefore, FVL homozygotes (and FVL pseudo-homozygotes) may benefit from therapeutically correcting APC resistance by replacing FVL protein with normal FV.

It has also been observed in accordance with the present disclosure that for FVL heterozygotes, FV pLOF heterozygotes do not show increased risk of VTE or major bleeding and in vitro depletion of normal FV down to 50% does not result in APC resistance. Therefore, selective depletion of FVL in heterozygotes may eliminate excess risk with no increase in bleeding risk. In addition, FVL heterozygotes in the top 10% of VTE PRS have the same risk of VTE as FVL homozygotes and could be a target population.

For purposes of the present disclosure, any particular subject, such as a human, can be categorized as having one of three FVL genotypes: i) FV reference; ii) heterozygous for an FVL variant nucleic acid molecule; or iii) homozygous for an FVL variant nucleic acid molecule. A subject is FV reference when the subject does not have a copy of an FVL variant nucleic acid molecule. A subject is heterozygous for an FVL variant nucleic acid molecule when the subject has a single copy of an FVL variant nucleic acid molecule. A subject is homozygous for an FVL variant nucleic acid molecule when the subject has two copies of an FVL variant nucleic acid molecule.

In any of the embodiments described herein, the FVL variant genomic nucleic acid molecule includes a guanine to adenine substitution resulting in an Arg506GIn alteration in the FV protein using the nucleotide sequence of the FV reference genomic nucleic acid molecule in the GRCh38/hg38 human genome assembly at chromosome 1 (i.e., positions 169,511,951-169,586,588) (see, ENSG00000198734, ENST00000367797 annotated in the in the Ensembl database (URL: world wide web at “https://useast.ensembl.org/Homo_sapiens/Gene/Summary?g=ENSG00000198734;r=1:169511951-169586588;transcript=ENST00000367797.9”)) as a reference sequence. The sequences provided in these transcripts for the FV and FVL genomic nucleic acid molecule are only exemplary sequences. Other sequences for the FV and FVL genomic nucleic acid molecule are also possible.

The present disclosure provides methods of reducing the risk of VTE in a subject undergoing anti-coagulant treatment. The methods comprise determining or having determined whether the subject is heterozygous or homozygous for FVL. In some embodiments, the determination is carried out by performing or having performed a sequence analysis on a biological sample obtained from the subject to determine if the subject has a genotype comprising FVL. In a subject that is heterozygous for FVL, the methods further comprise selectively depleting FVL. In a subject that is homozygous for FVL, the methods comprise replacing FVL in the genome with a functioning Factor V gene.

In some embodiments, the subject that is heterozygous for FVL also comprises an FV gene that encodes an FV variant polypeptide having a partial loss-of-function, a complete loss-of-function, a predicted partial loss-of-function, or a predicted complete loss-of-function (i.e., pLOF). Such a subject having one copy of FVL and an FV pLOF in the opposite haplotype is termed an FVL pseudo-homozygote, and may receive the same treatment as a subject that is FVL homozygous. In some embodiments, the FV pLOF variant nucleic acid molecule may result in decreased or aberrant expression or activity of FV mRNA or polypeptide. In some embodiments, the FV pLOF variant nucleic acid molecule may be associated with a reduced in vitro response to FV ligands compared to reference FV. In some embodiments, the FV pLOF variant nucleic acid molecule is a splice-site variant, a stop-gain variant, a start-loss variant, a stop-loss variant, a frameshift variant, an in-frame indel variant, or a variant that encodes a truncated FV variant polypeptide. In some embodiments, the FV pLOF variant nucleic acid molecule is a missense variant nucleic acid molecule. In some embodiments, the FV pLOF variant nucleic acid molecule comprises a single nucleotide polymorphism (SNP). In some embodiments, the FV pLOF variant nucleic acid molecule comprises a variation in a coding region. In some embodiments, the FV pLOF variant nucleic acid molecule does not comprise a variation in a non-coding region, except for a splice acceptor region (two bases before the start of any exon except the first). In some embodiments, the FV pLOF variant nucleic acid molecule results or is predicted to result in a premature truncation of an FV polypeptide compared to the reference FV. In some embodiments, the FV pLOF variant nucleic acid molecule is a variant that is predicted to be damaging to the protein function (and hence, in this case, protective to the human) by in vitro prediction algorithms such as Polyphen, SIFT, or similar algorithms. In some embodiments, the FV pLOF variant nucleic acid molecule is a variant that causes or is predicted to cause a nonsynonymous amino acid substitution in an FV nucleic acid molecule and whose allele frequency is less than 1/100 alleles in the population from which the subject is selected. In some embodiments, the FV pLOF variant nucleic acid molecule is a rare missense variant (allele frequency<0.1%; or 1 in 1,000 alleles), or a splice-site, stop-gain, start-loss, stop-loss, frameshift, or in-frame indel, or other frameshift FV variant. Therefore, FVL pseudo-homozygotes may benefit from therapeutically correcting APC resistance by replacing FVL protein with normal FV.

In some embodiments, selectively depleting FVL comprises administering to the subject an inhibitory nucleic acid molecule that hybridizes to an FVL nucleic acid molecule. In some embodiments, the inhibitory nucleic acid molecule comprises an antisense nucleic acid molecule, a small interfering RNA (siRNA), and/or a short hairpin RNA (shRNA). In some embodiments, the inhibitory nucleic acid molecule comprises an siRNA. In some embodiments, the inhibitory nucleic acid molecule comprises an antisense nucleic acid molecule. Such inhibitory nucleic acid molecules can be designed to target any region of an FVL nucleic acid molecule. In some embodiments, the antisense RNA, siRNA, or shRNA hybridizes to a sequence within an FVL genomic nucleic acid molecule or mRNA molecule and decreases expression of the FVL polypeptide in a cell in the subject.

The inhibitory nucleic acid molecules can comprise RNA, DNA, or both RNA and DNA. The inhibitory nucleic acid molecules can also be linked or fused to a heterologous nucleic acid sequence, such as in a vector, or a heterologous label. For example, the inhibitory nucleic acid molecules can be within a vector or as an exogenous donor sequence comprising the inhibitory nucleic acid molecule and a heterologous nucleic acid sequence. The inhibitory nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3XFLAG, 6XHis or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The inhibitory nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The inhibitory nucleic acid molecules can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O—, S—, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙNOCH₃, —O(CH₂)ₙNH₂, —O(CH₂)ₙCH₃, —O(CH₂)ₙONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

In some embodiments, the antisense nucleic acid molecules are gapmers, whereby the first one to seven nucleotides at the 5′ and 3′ ends each have 2′-methoxyethyl (2′-MOE) modifications. In some embodiments, the first five nucleotides at the 5′ and 3′ ends each have 2′-MOE modifications. In some embodiments, the first one to seven nucleotides at the 5′ and 3′ ends are RNA nucleotides. In some embodiments, the first five nucleotides at the 5′ and 3′ ends are RNA nucleotides. In some embodiments, each of the backbone linkages between the nucleotides is a phosphorothioate linkage.

In some embodiments, the siRNA molecules have termini modifications. In some embodiments, the 5′ end of the antisense strand is phosphorylated. In some embodiments, 5′-phosphate analogs that cannot be hydrolyzed, such as 5′-(E)-vinyl-phosphonate are used.

In some embodiments, the siRNA molecules have backbone modifications. In some embodiments, the modified phosphodiester groups that link consecutive ribose nucleosides have been shown to enhance the stability and in vivo bioavailability of siRNAs The non-ester groups (—OH, ═O) of the phosphodiester linkage can be replaced with sulfur, boron, or acetate to give phosphorothioate, boranophosphate, and phosphonoacetate linkages. In addition, substituting the phosphodiester group with a phosphotriester can facilitate cellular uptake of siRNAs and retention on serum components by eliminating their negative charge.

In some embodiments, the siRNA molecules have sugar modifications. In some embodiments, the sugars are deprotonated (reaction catalyzed by exo-and endonucleases) whereby the 2′-hydroxyl can act as a nucleophile and attack the adjacent phosphorous in the phosphodiester bond. Such alternatives include 2′-O-methyl, 2′-O-methoxyethyl, and 2′-fluoro modifications.

In some embodiments, the siRNA molecules have base modifications. In some embodiments, the bases can be substituted with modified bases such as pseudouridine, 5′-methylcytidine, N6-methyladenosine, inosine, and N7-methylguanosine.

In some embodiments, the siRNA molecules are conjugated to lipids. Lipids can be conjugated to the 5′ or 3′ termini of siRNA to improve their in vivo bioavailability by allowing them to associate with serum lipoproteins. Representative lipids include, but are not limited to, cholesterol and vitamin E, and fatty acids, such as palmitate and tocopherol.

In some embodiments, a representative siRNA has the following formula:

Sense:
mN*mN*/i2FN/mN/i2FN/mN/i2FN/mN/i2FN/mN/i2FN/mN/
i2FN/mN/i2FN/mN/i2FN/*mN*/32FN/
Antisense:
/52FN/*/i2FN/*mN/i2FN/mN/i2FN/mN/i2FN/mN/i2FN/mN/
i2FN/mN/i2FN/mN/i2FN/mN/i2FN/mN*N*N

wherein: “N” is the base; “2F” is a 2′-F modification; “m” is a 2′-O-methyl modification, “I” is an internal base; and “*” is a phosphorothioate backbone linkage.

In any of the embodiments described herein, the inhibitory nucleic acid molecules may be administered, for example, as one-to two-hour i.v. infusions or s.c. injections. In any of the embodiments described herein, the inhibitory nucleic acid molecules may be administered at dose levels that range from about 50 mg to about 900 mg, from about 100 mg to about 800 mg, from about 150 mg to about 700 mg, or from about 175 mg to about 640 mg (2.5 to 9.14 mg/kg; 92.5 to 338 mg/m²—based on an assumption of a body weight of 70 kg and a conversion of mg/kg to mg/m² dose levels based on a mg/kg dose multiplier value of 37 for humans).

The present disclosure also provides vectors comprising any one or more of the inhibitory nucleic acid molecules. In some embodiments, the vectors comprise any one or more of the inhibitory nucleic acid molecules and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

The present disclosure also provides compositions comprising any one or more of the inhibitory nucleic acid molecules. In some embodiments, the composition is a pharmaceutical composition. In some embodiments, the compositions comprise a carrier and/or excipient. Examples of carriers include, but are not limited to, poly (lactic acid) (PLA) microspheres, poly (D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

In some embodiments, a nuclease agent that induces one or more nicks or double-strand breaks at a recognition sequence(s) or a DNA-binding protein that binds to a recognition sequence within an FVL genomic nucleic acid molecule can be used to selectively deplete FVL. The recognition sequence can be located within a coding region of the FVL gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the FVL gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

Suitable nuclease agents and DNA-binding proteins for use herein include, but are not limited to, zinc finger protein or zinc finger nuclease (ZFN) pair, Transcription Activator-Like Effector (TALE) protein or Transcription Activator-Like Effector Nuclease (TALEN), or Clustered Regularly Interspersed Short Palindromic Repeats (CRISPR)/CRISPR-associated (Cas) systems. The length of the recognition sequence can vary, and includes, for example, recognition sequences that are about 30-36 bp for a zinc finger protein or ZFN pair, about 15-18 bp for each ZFN, about 36 bp for a TALE protein or TALEN, and about 20 bp for a CRISPR/Cas guide RNA.

In some embodiments, CRISPR/Cas systems can be used to modify an FVL genomic nucleic acid molecule within a cell. The methods and compositions disclosed herein can employ CRISPR-Cas systems by utilizing CRISPR complexes (comprising a guide RNA (gRNA) complexed with a Cas protein) for site-directed cleavage of FVL nucleic acid molecules.

Cas proteins generally comprise at least one RNA recognition or binding domain that can interact with gRNAs. Cas proteins can also comprise nuclease domains (such as, for example, DNase or RNase domains), DNA binding domains, helicase domains, protein-protein interaction domains, dimerization domains, and other domains. Suitable Cas proteins include, for example, a wild type Cas9 protein and a wild type Cpf1 protein (such as, for example, FnCpf1). A Cas protein can have full cleavage activity to create a double-strand break in an FVL genomic nucleic acid molecule or it can be a nickase that creates a single-strand break in an FVL genomic nucleic acid molecule. Additional examples of Cas proteins include, but are not limited to, Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csyl, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof. In some embodiments, a Cas system, such as Cas12a, can have multiple gRNAs encoded into a single crRNA. Cas proteins can also be operably linked to heterologous polypeptides as fusion proteins. For example, a Cas protein can be fused to a cleavage domain, an epigenetic modification domain, a transcriptional activation domain, or a transcriptional repressor domain. Cas proteins can be provided in any form. For example, a Cas protein can be provided in the form of a protein, such as a Cas protein complexed with a gRNA. Alternately, a Cas protein can be provided in the form of a nucleic acid molecule encoding the Cas protein, such as an RNA or DNA.

In some embodiments, targeted genetic modifications of FVL genomic nucleic acid molecules can be generated by contacting a cell with a Cas protein and one or more gRNAs that hybridize to one or more gRNA recognition sequences within a target genomic locus in the FVL genomic nucleic acid molecule. The gRNA recognition sequence can include or be proximate to the start codon of an FVL genomic nucleic acid molecule or the stop codon of an FVL genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon.

The gRNA recognition sequences within a target genomic locus in an FVL genomic nucleic acid molecule are located near a Protospacer Adjacent Motif (PAM) sequence, which is a 2-6 base pair DNA sequence immediately following the DNA sequence targeted by the Cas9 nuclease. The canonical PAM is the sequence 5′-NGG-3′ where “N” is any nucleobase followed by two guanine (“G”) nucleobases. gRNAs can transport Cas9 to anywhere in the genome for gene editing, but no editing can occur at any site other than one at which Cas9 recognizes PAM. In addition, 5′-NGA-3′ can be a highly efficient non-canonical PAM for human cells. Generally, the PAM is about 2-6 nucleotides downstream of the DNA sequence targeted by the gRNA. The PAM can flank the gRNA recognition sequence. In some embodiments, the gRNA recognition sequence can be flanked on the 3′ end by the PAM. In some embodiments, the gRNA recognition sequence can be flanked on the 5′ end by the PAM. For example, the cleavage site of Cas proteins can be about 1 to about 10, about 2 to about 5 base pairs, or three base pairs upstream or downstream of the PAM sequence. In some embodiments (such as when Cas9 from S. pyogenes or a closely related Cas9 is used), the PAM sequence of the non-complementary strand can be 5′-NGG-31, where Nis any DNA nucleotide and is immediately 3′ of the gRNA recognition sequence of the non-complementary strand of the target DNA. As such, the PAM sequence of the complementary strand would be 5′-CCN-3′, where N is any DNA nucleotide and is immediately 5′ of the gRNA recognition sequence of the complementary strand of the target DNA.

A gRNA is an RNA molecule that binds to a Cas protein and targets the Cas protein to a specific location within an FVL genomic nucleic acid molecule. An exemplary gRNA is a gRNA effective to direct a Cas enzyme to bind to or cleave an FVL genomic nucleic acid molecule, wherein the gRNA comprises a DNA-targeting segment that hybridizes to a gRNA recognition sequence within the FVL genomic nucleic acid molecule. Exemplary gRNAs comprise a DNA-targeting segment that hybridizes to a gRNA recognition sequence present within an FVL genomic nucleic acid molecule that includes or is proximate to the start codon or the stop codon. For example, a gRNA can be selected such that it hybridizes to a gRNA recognition sequence that is located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the start codon or located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the stop codon. Suitable gRNAs can comprise from about 17 to about 25 nucleotides, from about 17 to about 23 nucleotides, from about 18 to about 22 nucleotides, or from about 19 to about 21 nucleotides. In some embodiments, the gRNAs can comprise 20 nucleotides.

The Cas protein and the gRNA form a complex, and the Cas protein cleaves the FVL genomic nucleic acid molecule. The Cas protein can cleave the nucleic acid molecule at a site within or outside of the nucleic acid sequence present in the FVL genomic nucleic acid molecule to which the DNA-targeting segment of a gRNA will bind. For example, formation of a CRISPR complex (comprising a gRNA hybridized to a gRNA recognition sequence and complexed with a Cas protein) can result in cleavage of one or both strands in or near (such as, for example, within 1, 2, 3, 4, 5, 6, 7, 8, 9, 10, 20, 50, or more base pairs from) the nucleic acid sequence present in the FVL genomic nucleic acid molecule to which a DNA-targeting segment of a gRNA will bind.

Such methods can result, for example, in an FVL genomic nucleic acid molecule in which a region of the FVL genomic nucleic acid molecule is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the FVL genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

In some embodiments, selectively depleting FVL comprises administering to the subject anti-FVL antibody, or antigen-binding fragment thereof.

In some embodiments, replacing FVL in the genome of the subject that is homozygous for FVL with a functioning Factor V gene comprises performing base-pair editing to replace the glutamine at position 506 of Factor V with an arginine. Base editing can be either DNA base editing or RNA base editing.

In some embodiments, the base-pair editing comprises DNA base editing. Base editing can be performed to make a targeted change in FVL to result in normal FV. The technology has been created to target and correct single point mutations. It involves the potent DNA-scanning and sequence-identification capabilities of CRISPR-Cas9 system (CRISPR: clustered regularly interspaced short palindromic repeats, Cas9: CRISPR-associated protein 9) and a deaminase enzyme that can remove an amino group from a nucleotide and thereby chemically alter the target DNA sequence. Two main classes of base editors have been developed to date: cytosine base editors (CBEs), which catalyze the conversion of C-G base pairs to T-A base pairs; and adenine base editors (ABEs), which catalyze A-T-to-G-C conversions. CBEs use cytidine deaminases to convert cytosines to uracils, which are read by the cell's DNA polymerases as thymine. Similarly, ABEs convert adenosines to inosines, which are interpreted as guanines by polymerases.

A component of base editors includes Cas9 nickase (nCas9). At one time, catalytically dead versions of Cas9 (dCas9, that could not introduce DNA double-stranded breaks) were used in the first-generation base editors and have been substituted with the Cas9 nickase (nCas9) version that are found nearly in all base editors. The nickase can only nick the complementary strand of the DNA. This version of Cas9 that has been modified through mutations in one of the two main amino acid residues responsible for the DNA cleavage activity of Cas9. Therefore, nCas9 can still pair with a gRNA and find the DNA sequence complementary to the gRNA spacer but can only nick one strand of the DNA.

Another component of base editors includes nucleoside deaminase. CBEs contain cytosine deaminases, while ABEs typically contain adenine deaminases These deaminases are fused to the nickase and can remove an amino group from a specific type of nucleoside. The Cas9-deaminase fusion protein complex is targeted to a specific DNA locus by a guide RNA (gRNA). Once the base editor binds to its target sequence, the deaminase can modify bases within the exposed non-target strand (NTS) of the target site. The area of the target locus in which bases can be modified is called the base editing window. Deamination of substrate nucleotides within the editing window initially generates uridine and inosine, creating a mismatched DNA base pair with the G or T, respectively, on the opposite, non-deaminated strand. Stable base editing outcomes require replacement of the unedited strand to install the corresponding A and C complementary nucleotides opposite the uridine or inosine, respectively. However, uracil and inosine intermediates are mutagenic, and DNA repair pathways have evolved in most organisms to remove these bases from genomic DNA. Uracil is rapidly excised from genomic DNA by uracil DNA N-glycosylase. If uracil excision occurs before installation of the complementary G-to-A conversion on the non-deaminated strand, the resulting site will often revert to the original sequence (or an undesired transversion mutation) through the base excision repair pathway. To increase the half-life of uracil at the target locus and consequently increase editing efficiency and purity, CBEs typically include uracil glycosylase inhibitor proteins (UGIs) that substantially increase editing yield and product purity. For ABEs, inhibition of MPG, the glycosylase thought to excise inosine from genomic DNA in eukaryotic cells, did not further increase already very high editing product purities, suggesting that inosine excision is much less efficient in mammalian cells than uracil excision. The resulting nick stimulates cellular repair of the non-deaminated strand, using the deaminated strand as a template for resynthesizing the nicked strand. Deamination of one strand and resynthesis of the complementary strand therefore results in editing of both target DNA strands to yield stable conversion of the target base pair.

CRISPR-Cas9 allow base editing, which is the direct, irreversible conversion of one base pair to another at a target genomic sequence without inducing a double-stranded DNA break. RNA is generally expressed only in specific cell types or at certain times. Hence, fewer off-target editing side effects are expected when using an editing system targeting RNA than during genome sequence editing. Furthermore, incorrect RNA editing does not affect fetal development because the genome sequence is not affected, and mutated RNAs are quickly degraded if treatment is discontinued. Therefore, RNA editing is more flexible than genome editing for clinical uses.

RNA editing is a technique to restore RNA sequences in order to treat genetic diseases caused by point mutations. It describes the alteration of an RNA sequence by introducing or removing nucleotides from an RNA or by changing the character of a nucleobase by deamination. For therapeutic purposes, the two types of deamination reactions, converting cytidine to uridine and adenosine to inosine are considered most useful and enable endogenous RNA modulation beyond RNA overexpression or knockdown.

A-to-I editing is mediated by adenosine deaminases acting on RNA (ADARs). These enzymes bind double-stranded or structured regions in RNAs via their double-stranded RNA-binding domains. An adenosine that is typically located within the double-stranded region is then recognized by the catalytic deaminase domain and converted to inosine. Substrate-binding and deamination occur with relatively low specificity, leading to abundant and widespread editing in vivo. The RNA editing machinery relies on two critical components: complementary RNA sequences that can precisely bind to specified sequences (guide RNA) and deamination-editing enzyme/editors.

In some embodiments, replacing FVL in the genome of the subject that is homozygous for FVL with a functioning Factor V gene comprises replacing the FVL gene in the genome of the subject with a Factor V gene. In some embodiments, replacing FVL in the genome of the subject that is homozygous for FVL with a functioning Factor V gene comprises replacing the nucleic acids encoding the A1-A2 domain of the FVL in the genome of the subject with nucleic acids encoding the A1-A2 domain of Factor V.

Gene-insertion of full-length FV can be performed. Partial gene insertion can also be performed using A1-A2-[B-A3 junction] domains of FV. FV comprises six major domains, organized as A1-A2-B-A3-C1-C2. Activation by thrombin or FXa releases the B domain leading to formation of two fragments, FVa heavy chain (A1-A2 fragment) and light chain (A3-C1-C2 fragment). The fragments are held together by a single calcium atom. However, APC-cofactor function of FV for factor VIIIa (FVIIIa) inactivation depends on the C-terminal part of the B-domain (residues 1477-1545) that form the B-A3 junction. Gene-insertion can be performed with a construct comprising AAV packaged with the vector harboring the coding DNA sequence for the FV A1-A2-[B-A3 junction] domains.

For episomal delivery, the construct may be placed under a strong promoter to drive expression. For a gene insertion approach, the construct may be placed in, for example, the promoter-less REGV131LNP1265 vector. REGV131LNP1265 is a gene therapy medicinal product designed to use CRISPR/Cas9-based gene insertion technology to insert a promoter less human FV A1-A2-[B-A3 junction] DNA template into the ALB locus, resulting in ALB promoter-driven A1-A2-[B-A3 junction] protein expression and secretion into the plasma. The vector is composed of 2 components: i) an rAAV8 vector (REGV131), and ii) an LNP (LNP1265). REGV131 is an rAAV8 vector that displays liver tropism, containing a single-stranded bidirectional promoter less A1-A2-[B-A3 junction] DNA template. LNP1265 is a liver directed LNP composed of a proprietary ionizable lipid (LP000001), DSPC, cholesterol, and DMGPEG2k, which encapsulates: i) codon-optimized cas9 mRNA (ID: mRNA000042) encoding the Cas9 protein, and ii) sgRNA (ID: G009860) containing a 20-nucleotide sequence that is complementary to a target sequence in intron 1 of the human and cynomolgus monkey ALB genes.

In any of the embodiments disclosed herein, the methods may further comprise obtaining or having obtained a biological sample from the subject. In some embodiments, the FVL nucleic acid molecule is an FVL genomic nucleic acid molecule, or is an mRNA molecule produced therefrom, or is a cDNA molecule produced from the mRNA molecule.

In any of the embodiments disclosed herein, the VTE comprises deep vein thrombosis (DVT). In any of the embodiments disclosed herein, the VTE comprises pulmonary embolism (PE).

Detecting the presence or absence of an FVL nucleic acid molecule or FVL polypeptide in a biological sample from a subject and/or determining whether a subject has an FVL nucleic acid molecule or FVL polypeptide can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, the anti-coagulant treatment includes, but is not limited to, treatment with: i) a coumarin or indandione, such as warfarin; ii) a factor Xa inhibitor, such as rivaroxaban, apixaban, fondaparinux, edoxaban, and betrixaban; iii) a heparin, such as enoxaparin, dalteparin, tinzaparin, danaparoid, and heparin flush; and iv) a thrombin inhibitor, such as dabigatran, bivalirudin, and desirudin.

Administration of the anti-coagulant treatment can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients, or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in VTE, a decrease/reduction in the severity of VTE (such as, for example, a reduction or inhibition of development of VTE), a decrease/reduction in symptoms and disease-related effects, delaying the onset of symptoms and disease-related effects, reducing the severity of symptoms of disease-related effects, reducing the number of symptoms and disease-related effects, reducing the latency of symptoms and disease-related effects, an amelioration of symptoms and disease-related effects, reducing secondary symptoms, reducing secondary infections, preventing relapse to VTE, decreasing the number or frequency of relapse episodes, increasing latency between symptomatic episodes, increasing time to sustained progression, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of VTE development/progression (such as, for example, a complete or partial avoidance/inhibition or a delay), and an increased survival time of the affected host animal, following administration of a therapeutic protocol. Treatment of VTE encompasses the treatment of a subject already diagnosed as having any form of VTE at any clinical stage or manifestation, the delay of the onset or evolution or aggravation or deterioration of the symptoms or signs of VTE, and/or preventing and/or reducing the severity of VTE.

The present disclosure also provides methods of identifying a subject undergoing anti-coagulant treatment who is at risk of developing VTE. The methods comprise determining or having determined the presence or absence of FVL nucleic acid molecule in a biological sample obtained from the subject. When the subject has a nucleic acid molecule encoding FVL, then the subject has an increased risk of developing VTE. When the subject does not have a nucleic acid molecule encoding FVL, then the subject does not have an increased risk of developing VTE.

In some embodiments, the methods further comprise: i) selectively depleting FVL in a subject that is heterozygous for a nucleic acid molecule encoding FVL; or ii) replacing FVL in the genome of the subject that is homozygous for FVL with a functioning Factor V gene (as described herein).

The present disclosure also provides methods of detecting the presence or absence of an FVL nucleic acid molecule (i.e., a genomic nucleic acid molecule, an mRNA molecule, or a cDNA molecule produced from an mRNA molecule) in a biological sample from a subject. It is understood that gene sequences within a population and mRNA molecules encoded by such genes can vary due to polymorphisms such as single-nucleotide polymorphisms.

The biological sample can be derived from any cell, tissue, or biological fluid from the subject. The biological sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The biological sample used in the methods disclosed herein can vary based on the assay format, nature of the detection method, and the tissues, cells, or extracts that are used as the sample. A biological sample can be processed differently depending on the assay being employed. For example, when detecting any FVL nucleic acid molecule, preliminary processing designed to isolate or enrich the biological sample for the genomic DNA can be employed. A variety of techniques may be used for this purpose. When detecting the level of any FVL nucleic acid molecule, different techniques can be used enrich the biological sample with mRNA molecules. Various methods to detect the presence or level of an mRNA molecule or the presence of a particular variant genomic DNA locus can be used.

In some embodiments, detecting an FVL nucleic acid molecule in a subject comprises performing a sequence analysis on a biological sample obtained from the subject to determine whether an FVL genomic nucleic acid molecule in the biological sample, and/or an FVL mRNA molecule in the biological sample, and/or an FVL cDNA molecule produced from an mRNA molecule in the biological sample, is present in the sample. In some embodiments, the methods detect the FVL genomic nucleic acid molecule that comprises the genetic variation coding for FVL Arg506Gln, or an mRNA molecule produced therefrom, or a cDNA molecule produced from the mRNA molecule.

In some embodiments, the methods of detecting the presence or absence of an FVL nucleic acid molecule (such as, for example, a genomic nucleic acid molecule, an mRNA molecule, and/or a cDNA molecule produced from an mRNA molecule) in a subject comprise performing an assay on a biological sample obtained from the subject. The assay determines whether a nucleic acid molecule in the biological sample comprises a particular nucleotide sequence.

In some embodiments, the biological sample comprises a cell or cell lysate. Such methods can further comprise, for example, obtaining a biological sample from the subject comprising an FVL genomic nucleic acid molecule or mRNA molecule, and if mRNA, optionally reverse transcribing the mRNA into cDNA. Such assays can comprise, for example determining the identity of these positions of the particular FVL nucleic acid molecule. In some embodiments, the method is an in vitro method.

In some embodiments, the determining step, detecting step, or sequence analysis comprises sequencing at least a portion of the nucleotide sequence of the FVL genomic nucleic acid molecule, the FVL mRNA molecule, or the FVL cDNA molecule in the biological sample that comprises a genetic variation compared to the corresponding V5 reference molecule.

In some embodiments, the assay comprises sequencing the entire nucleic acid molecule. In some embodiments, only an FVL genomic nucleic acid molecule is analyzed. In some embodiments, only an FVL mRNA is analyzed. In some embodiments, only an FVL cDNA obtained from the FVL mRNA is analyzed.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the nucleic acid molecule in the sample is mRNA and the mRNA is reverse-transcribed into a cDNA prior to the amplifying step. In some embodiments, the nucleic acid molecule is present within a cell obtained from the subject.

In some embodiments, the assay comprises contacting the biological sample with a primer or probe, such as an alteration-specific primer or alteration-specific probe, that specifically hybridizes to an FVL genomic sequence, mRNA sequence, or cDNA sequence and not the corresponding FV reference sequence under stringent conditions and determining whether hybridization has occurred.

In some embodiments, the determining step, detecting step, or sequence analysis comprises: a) amplifying at least a portion of the FVL nucleic acid molecule that encodes the FVL polypeptide; b) labeling the amplified nucleic acid molecule with a detectable label; c) contacting the labeled nucleic acid molecule with a support comprising an alteration-specific probe; and d) detecting the detectable label.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising an FVL genomic nucleic acid molecule, mRNA molecule, or cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to FVL nucleic acid molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the FVL nucleic acid molecules disclosed herein. The primers described herein can be used to amplify any FVL nucleic acid molecule, or a fragment thereof.

In the context of the disclosure “specifically hybridizes” means that the probe or primer (such as, for example, the alteration-specific probe or alteration-specific primer) does not hybridize to a nucleic acid sequence encoding an FV reference genomic nucleic acid molecule, an FV reference mRNA molecule, and/or an FV reference cDNA molecule.

In some embodiments, the probes (such as, for example, an alteration-specific probe) comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3XFLAG, 6Xhis or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1

### General Methods

### Genotype Data

High coverage whole exome sequencing was performed as previously described (Dewey et al., Science, 2016, 354, 6319; and Van Hout et al., Nature, 2020, 586, 749-756) and as summarized below. NimbleGen probes (VCRome; for part of the GHS cohort) or a modified version of the xGen design available from Integrated DNA Technologies (IDT; for the rest of GHS and other cohorts) were used for target sequence capture of the exome. A unique 6 base pair (bp) barcode (VCRome) or 10 bp barcode (IDT) was added to each DNA fragment during library preparation to facilitate multiplexed exome capture and sequencing. Equal amounts of sample were pooled prior to exome capture. Sequencing was performed using 75 bp paired-end reads on Illumina v4 HiSeq 2500 (for part of the GHS cohort) or NovaSeq (for the rest of GHS and other cohorts) instruments. Sequencing had a coverage depth (i.e., number of sequence-reads covering each nucleotide in the target areas of the genome) sufficient to provide greater than 20× coverage over 85% of targeted bases in 96% of VCRome samples and 20× coverage over 90% of targeted bases in 99% of IDT samples. Data processing steps included sample de-multiplexing using Illumina software, alignment to the GRCh38 Human Genome reference sequence including generation of binary alignment and mapping files (BAM), processing of BAM files (e.g., marking of duplicate reads and other read mapping evaluations). Variant calling was performed using the GLNexus system (DOI: 10.1101/343970). Variant mapping and annotation were based on the GRCh38 Human Genome reference sequence and Ensembl v85 gene definitions using the snpEff software. The snpEff predictions that involve protein-coding transcripts with an annotated start and stop were then combined into a single functional impact prediction by selecting the most deleterious functional effect class for each gene. The hierarchy (from most to least deleterious) for these annotations was frameshift, stop-gain, stop-loss, splice acceptor, splice donor, stop-lost, in-frame indel, missense, other annotations. Predicted LOF genetic variants included: a) insertions or deletions resulting in a frameshift, b) insertions, deletions or single nucleotide variants resulting in the introduction of a premature stop codon or in the loss of the transcription start site or stop site, and c) variants in donor or acceptor splice sites. Missense variants were classified for likely functional impact according to the number of in silico prediction algorithms that predicted deleteriousness using SIFT (Adzhubei et al., Nat. Methods, 2010, 7, 248-9) and Polyphen2_HVAR (Adzhubei et al., Nat. Methods, 2010, 7, 248-9), LRT (Chun et al., Genome Res., 2009, 19, 1553-61) and MutationTaster (Schwarz et al., Nat. Methods, 2010, 7, 575-6). For each gene, the alternative allele frequency (AAF) and functional annotation of each variant determined inclusion into these gene burden exposures: 1) pLOF variants with AAF<1%; 2) pLOF or missense variants predicted deleterious by 5/5 algorithms with AAF<1%.

### Association Analysis of FVL With VTE and Major Bleeding

Genetic association studies were performed in individuals of African or Admixed African, Admixed American, European, and East and South Asian ancestry. Association between FVL mutation (rs6025) and phenotypes were tested by fitting a firth bias-corrected logistic (for binary traits) regression model adjusted for a polygenic score that approximates a genomic kinship matrix using Regenie v3.2.7 (Mbatchou et al., bioRxiv, 2020, 06.19.162354). Analyses were stratified by ancestry and adjusted for age, age2, experimental batch-related covariates, 10 common variant-derived principal components, and 20 rare variant-derived principal components. Results across cohorts for each variant-phenotype association were combined using fixed effects inverse variance weighted meta-analysis.

### Participating Cohorts

The United Kingdom Biobank (UKB) (Bycroft et al., Nature, 2018, 562, 203-209; and Van Hout et al., Nature, 2020, 586, 749-756) is a population-based cohort study of people aged between 40 and 69 years recruited through 22 testing centers in the UK between 2006-2010. Whole-exome sequencing data of the participants and their phenotype data (VTE: 453,253 individuals, major bleeding: 324,471 individuals) were included from UKB. The MyCode Community Health Initiative cohort from the Geisinger Health System (GHS) (Carey et al., Genet Med., 2016, 18, 906-13) study is a health system-based cohort of patients from Central and Eastern Pennsylvania (USA) recruited in 2007-2019. A total of 161,990 individuals for VTE, 158,858 individuals for major bleeding, and their whole-exome sequencing data were included. The Malmö Diet and Cancer Study (MDCS) is a cohort study based in Malmo, Sweden (Berglund et al., J. Intern. Med., 1993, 233, 45-51). Whole-exome sequencing data and phenotype data (28,948 individuals for VTE and 18,977 individuals for major bleeding) were included from this cohort. The University of Pennsylvania Medicine BioBank (PMBB) is a health system-based cohort based in Pennsylvania. Whole-exome sequencing data and phenotype data on 39,409 for VTE and 38,598 for major bleeding phenotypes were included from PMBB. Mount Sinai's BioMe Personalized Medicine Cohort (SINAI) (Gottesman et al., Genet. Med., 2013, 15, 761-771) included individuals with whole exome sequencing data and available phenotypic data (VTE: 27,784, severe bleeding: 25,133). The Indiana University School of Medicine Biobank (INDIANA) biobank included whole-exome sequencing data VTE phenotype data on 5,684 individuals. INDIANA was excluded from major bleeding phenotype association analysis as the cohort includes patients with liver disease. The MAYO-CLINIC is a hospital-based electronic health record database (Olson et al., BMJ, 2019, 9: e032707) with genotypic and phenotypic data on 112,829 individuals for VTE and 83,016 individuals for major bleeding. The UCLA is a hospital-based electronic health record data that provided genotypic and phenotypic data on 37,177 individuals for VTE and 28,159 individuals for major bleeding. COLORADO is a hospital-based electronic health record database with genotypic and phenotypic data on 47,209 individuals for VTE and 33,149 individuals for major bleeding.

### Phenotype Definitions

Disease outcomes were defined according to the International Classification of Diseases, Ninth and Tenth Revision (ICD-9 and ICD-10) using EHRs and self-reports when available. ICD-9 and ICD-10 codes were mapped to Phe9 and Phe10 codes (phecodes) and combined into single variables as described in Table 1.

TABLE 1
Definitions of disease outcomes in UKB, GHS, SINAI,
UPENN-PMBB, MALMO and Indiana-Chalasani

| | | Control |
|---|---|---|
| Disease outcome | Case definition | definition |
| Venous | Phe10: | |
| thromboembolism | O225, O223, O871, O873, I636, I676, | |
| | I82, I81, I80, K645, I870, G08, I26, | |
| | O882 | |
| | UKB f20002: | |
| | 1068, 1093, 1094 | |
| | UKB Diagnosis by Doctor: | |
| | 6152_Blood_clot_DVT_bronchitis_{—} | |
| | emphysema_asthma_rhinitis_{—} | |
| | eczema_allergy_diagnosed_by_{—} | |
| | doctor_0_5_Blood_clot_in_the_{—} | |
| | leg_DVT, 6152_Blood_clot_DVT_{—} | |
| | bronchitis_emphysema_asthma_{—} | |
| | rhinitis_eczema_allergy_{—} | |
| | diagnosed_by_doctor_0_7_Blood_{—} | |
| | clot_in_the_lung | |
| Major bleeding | Phe10: | Phe10: |
| | P51, P52, I60, I61, I62, K920, K921, | K922, D691 |
| | M20, I690, I691, I692 | |
| | UKB 20002: | |
| | 1083, 1086, 1491 | |

aPTT Assay

The aPTT in plasma was determined using a STart4 Hemostasis Analyzer. Briefly, a total of 50 μl of plasma sample was added to a STart Cuvette and incubated at 37° C. for 1 minute. 50 μL of aPTT-XL (EA) was added for a 5-minute incubation followed by the addition of 50 μL of 20 mM calcium chloride solution to start the reaction. Average clotting times from duplicate measurements were reported.

### TGA-EA Assay

Thrombin generation profiles for intrinsic pathway activity were determined using a Calibrated Automated Thrombogram platform using EA. 60 μL of plasma sample was added to a well of an Immulon II HB U Bottom Microplate and incubated at 37° C. for 15 minutes; samples from the control group were also included for baseline measurement. Thrombin generation was then induced by addition of 0.167 μM aPTT-XL EA pre-diluted in MP reagent (intrinsic pathway-triggered activity). After incubation for 30 minutes at 37° C., 15 μl of pre-warmed Fluosubstrate prepared in Fluo-buffer was added to the wells before a 90 minute reading of the Immulon II HB U Bottom Microplate. The measured real-time thrombin concentration values were plotted against time to yield a thrombogram profile for each antibody concentration tested. The lag time, peak thrombin, and ETP were determined from each thrombogram.

### Example 2

### Factor V Leiden (FVL) is a Common Variant Associated With Large Effect Increase in VTE Risk

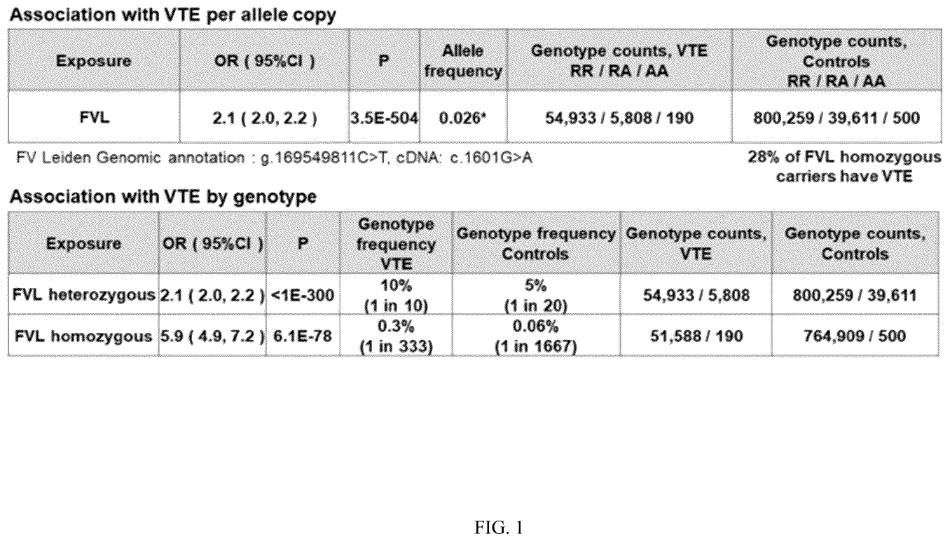

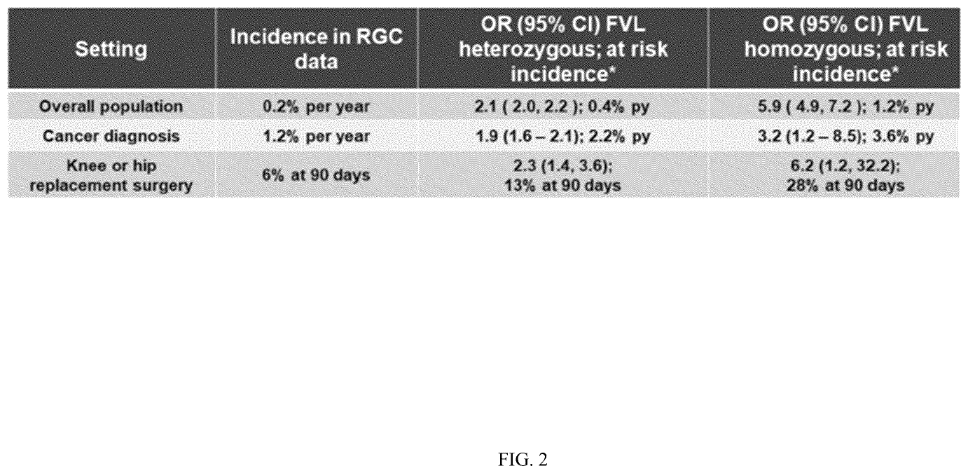

Whole exome sequencing data were analyzed for association of FVL with VTE in up to 904,792 participants from the Geisinger Heath System MyCode Community Health Initiative study (GHS), Malmö Diet and Cancer Study (MDCS), the UK Biobank (UKB) and the University of Pennsylvania Penn Medicine Biobank (UPENN-PMBB). FVL is a common variant with allele frequency of 2.6% and is associated with 2-fold higher risk of VTE (see, FIG. 1). While individuals heterozygous for FVL are at 2-fold higher risk, homozygotes are at approximately 6-fold higher risk of VTE. The incidence of VTE in overall population is 0.2% per year. However, the risk is amplified in patients with cancer (1.2% per year) and in individuals that undergo knee or hip replacement surgeries (6% at 90 days). Presence of the FVL mutation further increases VTE risk (see, FIG. 2). Heterozygous FVL individuals in the overall population have double the incidence (0.4% per year), which further increases to 2.2% in presence of cancer and 13% at 90 days post knee or hip replacement surgeries. Individuals homozygous for FVL mutation have 6 times more incidence of VTE (1.2% per year) compared to non-carriers in the overall population which is amplified to 3.6% per year in presence of cancer and 28% at 90 days after hip or knee replacement surgery.

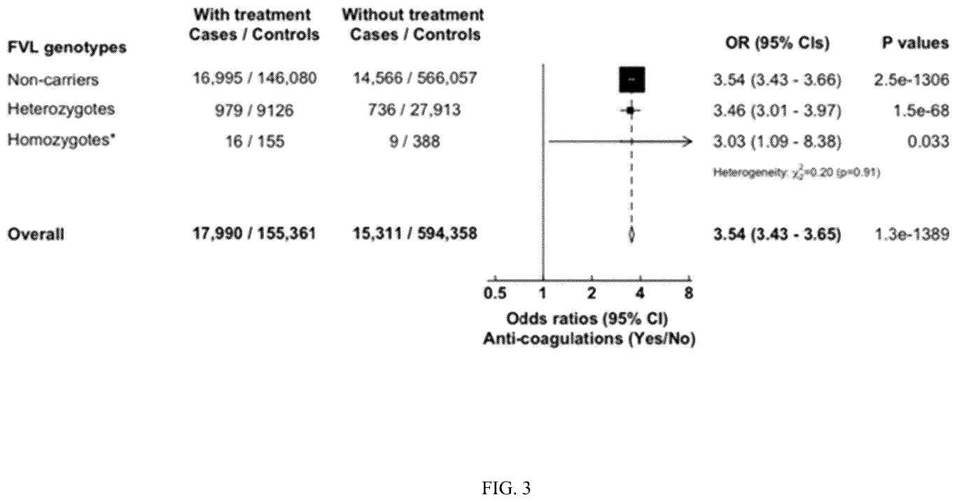

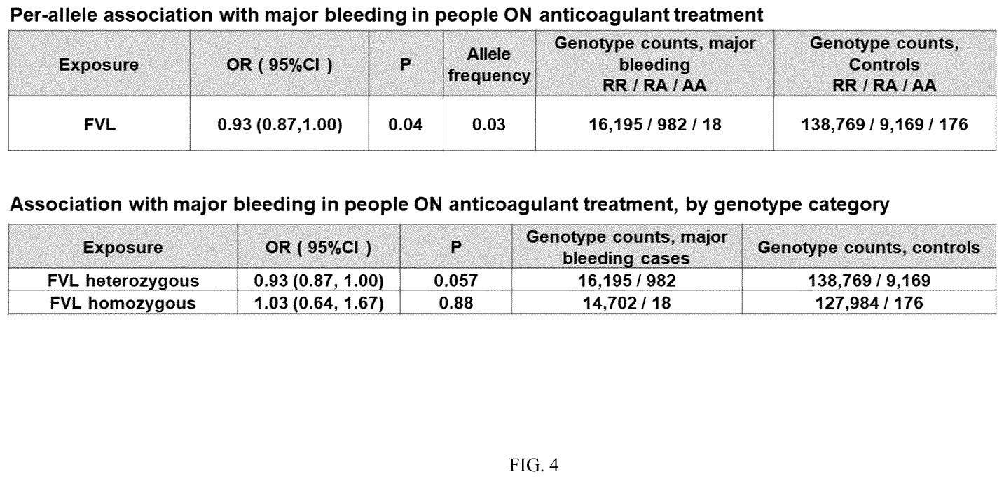

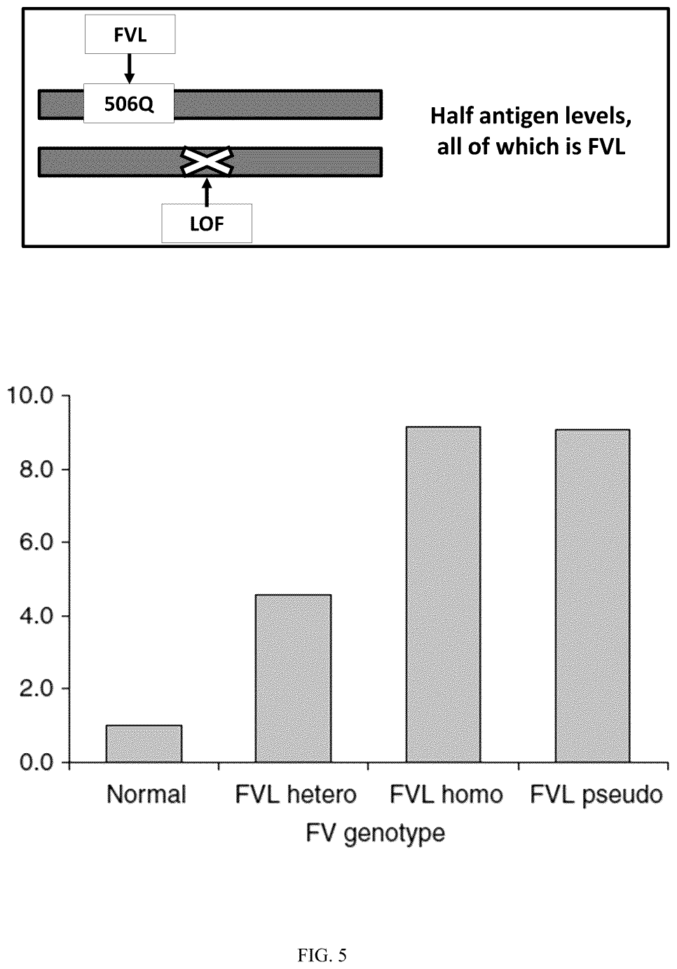

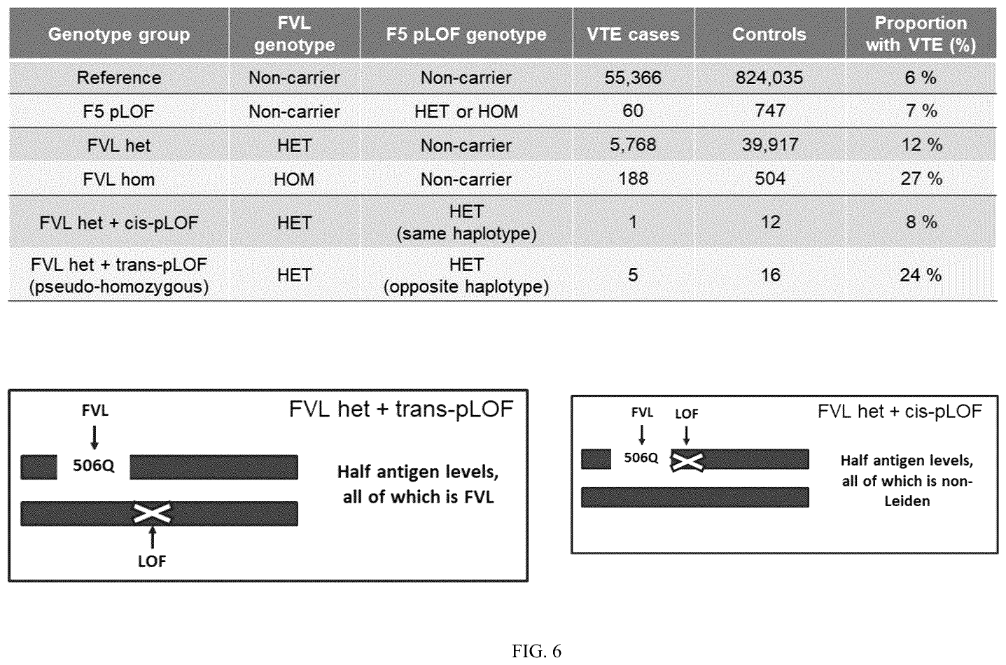

Although FVL mutation is pro-thrombotic, carriers have markedly increased risk of major bleeding when treated with anticoagulants compared to untreated carriers, but the bleeding risk is approximately the same as non-carriers of FVL (see, FIG. 3). FIG. 4 shows that the risk of bleeding is the same in carriers and non-carriers on anticoagulant treatment. FVL pseudo-homozygosity, which is FVL heterozygotes with loss of function variant in opposite haplotype (see, FIG. 5), is a natural model for partial knockdown of FVL. These individuals, therefore, have 50% FV antigen, all of which is FVL protein, and no FV wild type protein due to the presence of the pLOF variants in the opposite haplotype (in trans position). Literature suggests that FVL pseudohomozygous individuals have the same level of APC resistance as the individuals homozygous for FVL. The data presented herein (see, FIG. 6) shows that proportion of individuals with VTE is similar between pseudo-homozygous and homozygous FVL individuals, while individuals that have FVL and pLOFs in the same haplotype (in cis position) have risk of VTE that is similar to individuals that do not carry FVL mutation. This is because they do not make FVL protein due to the presence of pLOF variants in the same haplotype, but have a functional FV wild type protein (50% antigen level) coded by the opposite haplotype.

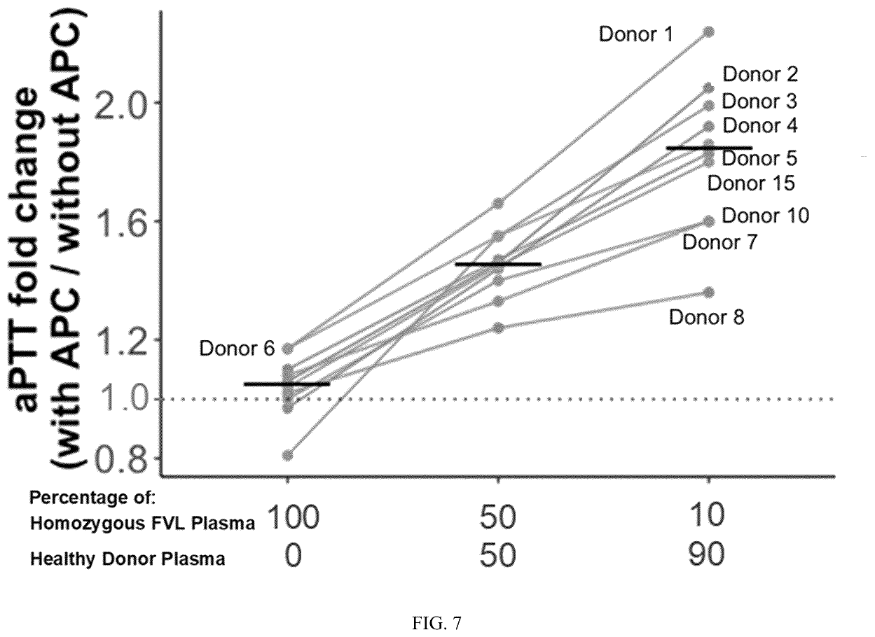

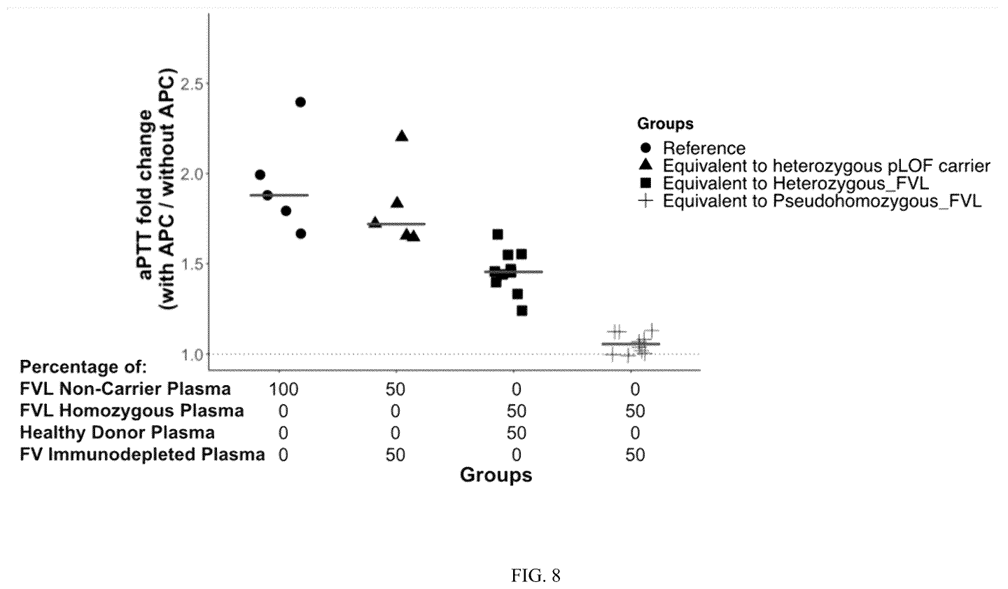

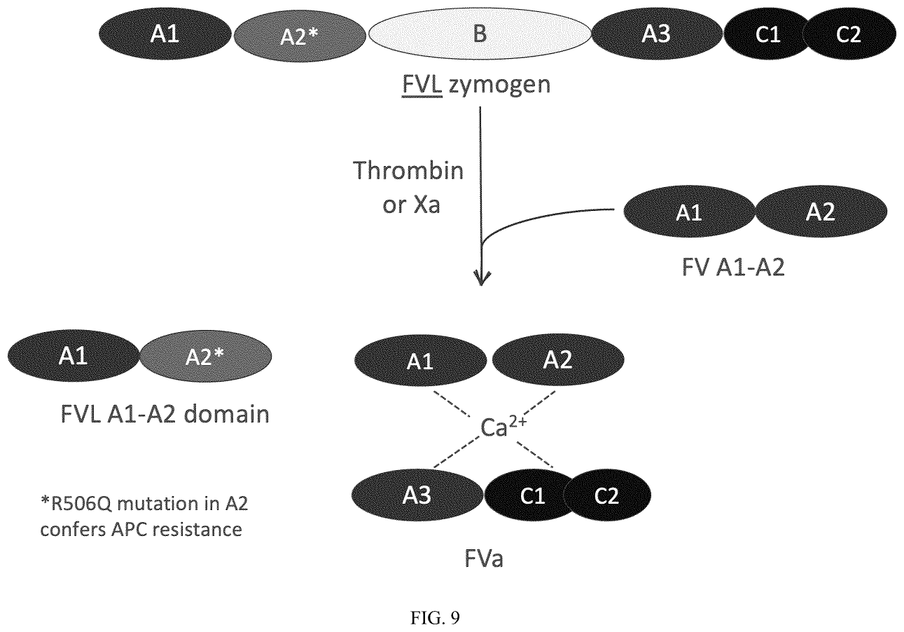

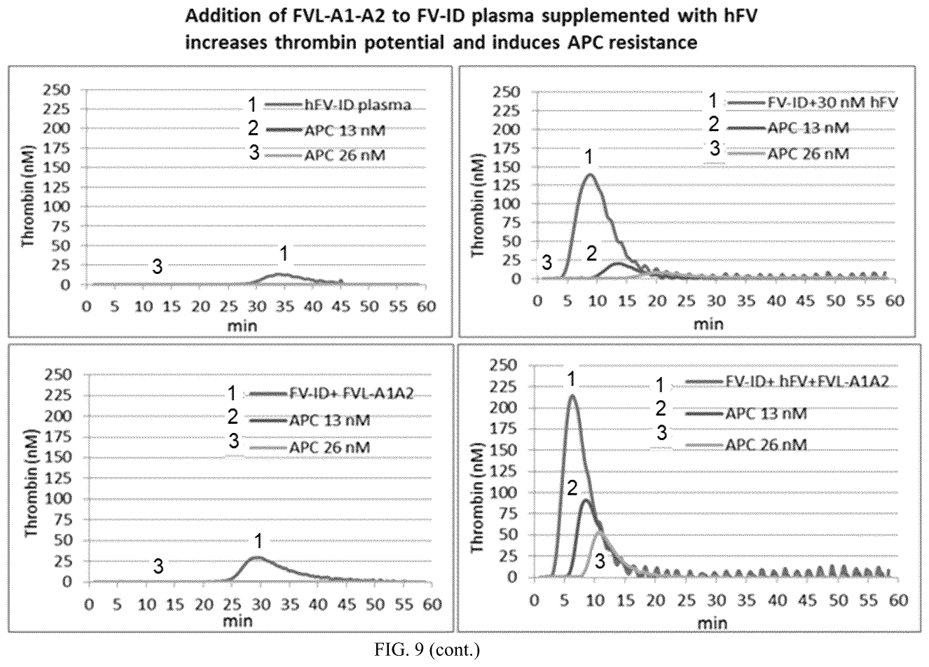

To determine if APC resistance can be corrected in individuals with FVL mutation, human plasma samples from homozygous FVL individuals (n=10) and non-FVL carriers were obtained from Amish individuals (not on anticoagulant treatment) through collaboration with University of Maryland. Healthy non-FVL plasma and FV immunodepleted plasma were purchased from Precision Biologic. Diluting down FVL homozygous plasma to 1% with FV immunodepleted plasma did not correct for APC resistance (see, FIG. 9). However, at 99% depletion, thrombin generation is reduced but FV depletion to that level is expected to result in significant major bleeding risk. Dilution of homozygous FVL plasma to 10% with healthy plasma containing normal FV ameliorated APC resistance (see, FIG. 7). FIG. 8 shows expected APC resistance per genotypes. Reducing FVL to 50% (diluting with FV immunedepleted plasma) does not ameliorate APC resistance, while diluting down homozygous FVL plasma with healthy plasma (containing normal FV) to 50% shows partly corrected APC resistance, as expected for individuals heterozygous for the FVL mutation. FIG. 8 also shows that FVL non-carrier plasma (one that does not contain FVL mutation) diluted down to 50% with FV immunodepleted plasma doesn't affect APC sensitivity, demonstrating that wild type FV even at 50% maintains APC sensitivity, in absence of the FVL mutation. The findings also confirmed that wild-type FV is needed to correct for APC resistance in individuals homozygous for FVL mutation.

Domain substitution of mutated FVL A1-A2 domain with wild-type FV A1-A2 domain during FV activation is depicted in FIG. 9. FV is activated upon cleavage of both the N- and C-terminals of the B-domain by thrombin or FXa, giving rise to an ionically held A1-A2 to Ca2+ to A3-C1-C2 complex. During the generation of this complex, the mutated FVL A1-A2 domain can be replaced with the wild-type FV A1-A2 domain. A thrombogram measuring the amount of thrombin generated over 60 minutes with 0, 13 or 26 nM of APC following extrinsic-mediated pathway activation was conducted in human FV-immunodepleted (hFV-ID) plasma (see, FIG. 9, upper left panel). Addition of a normal concentration of 30 nM of human FV to the hFV-ID plasma restored thrombin generation in the absence of APC; addition of APC showed expected anticoagulant effect on thrombin generation (see, FIG. 9, upper right panel). Use of the mutated FVL A1-A2 domain in FV-ID plasma had little effect on augmenting thrombin generation in plasma immunodepleted of FV (see, FIG. 9, lower left panel). Addition of mutated FVL A1-A2 domain in FV-ID plasma with normal level of hFV resulted in augmented thrombin generation in the absence of APC; addition of APC reduced but did not completely abolish thrombin generation, suggesting APC resistance due to FVL A1-A2 domain substitution with the wild-type FV A1-A2 domain (see, FIG. 9, lower right panel).

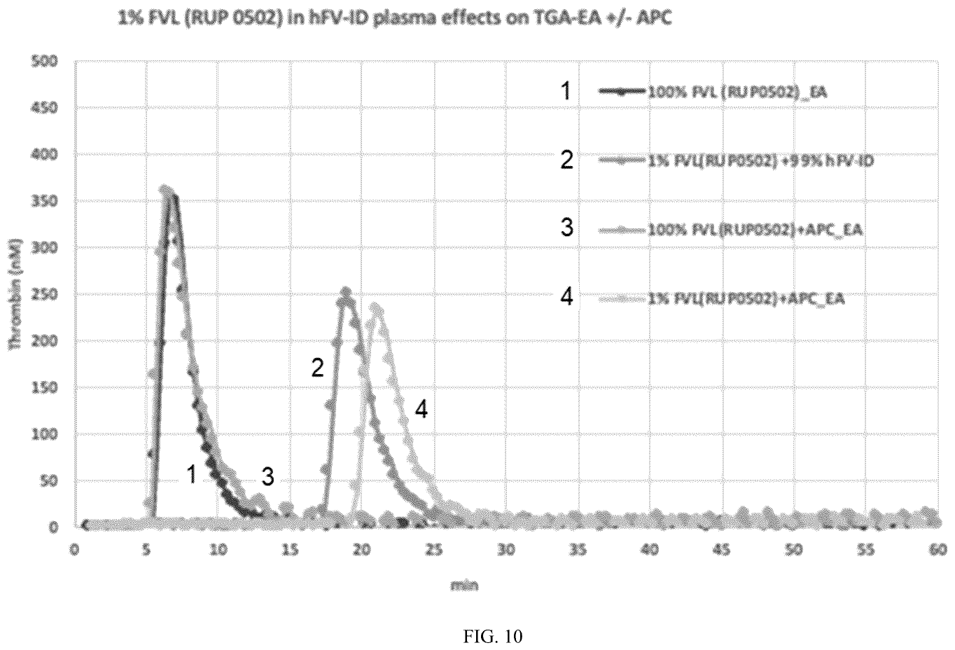

Plasma from an FVL homozygous individual (RU P0502) treated with APC showed no change to thrombin generation, indicating APC resistance (Brown vs Gray tracings) (see, FIG. 10). Once the FVL plasma was diluted to 1% with FV-immunodepleted plasma, the thrombin curve (Orange) shifted downward and to the right, indicative of reduced coagulation potential. Once the 1% FVL plasma was treated with APC (Yellow curve), there was a slight but not dramatic rightward shift which is suggestive of APC resistance in the diluted FVL plasma.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of reducing the risk of venous thromboembolism (VTE) in a subject undergoing anticoagulant treatment, the method comprising:
determining or having determined whether the subject is heterozygous or homozygous for Factor V Leiden (FVL) by performing or having performed a sequence analysis on a biological sample obtained from the subject to determine if the subject has a genotype comprising FVL; and
i) selectively depleting FVL in a subject that is heterozygous for FVL; or
ii) replacing FVL in the genome of the subject that is homozygous for FVL with a functioning Factor V gene.

2. The method of claim 1, wherein selectively depleting FVL comprises administering to the subject an inhibitory nucleic acid molecule that hybridizes to an FVL nucleic acid molecule.

3. The method of claim 2, wherein the inhibitory nucleic acid molecule comprises an antisense nucleic acid molecule, a small interfering RNA (siRNA), and/or a short hairpin RNA (shRNA).

4. The method of claim 3, wherein the inhibitory nucleic acid molecule comprises an siRNA or an antisense nucleic acid molecule.

5. (canceled)

6. The method of claim 1, wherein selectively depleting FVL comprises administering to the subject an anti-FVL antibody, or antigen-binding fragment thereof.

7. The method of claim 1, wherein replacing FVL in the genome of the subject that is homozygous for FVL with a functioning Factor V gene comprises performing base-pair editing to replace the glutamine at position 506 of Factor V with an arginine.

8. The method of claim 1, wherein replacing FVL in the genome of the subject that is homozygous for FVL with a functioning Factor V gene comprises replacing the FVL gene in the genome of the subject with a Factor V gene.

9. The method of claim 1, wherein replacing FVL in the genome of the subject that is homozygous for FVL with a functioning Factor V gene comprises replacing the nucleic acids encoding the A1-A2 domain of the FVL in the genome of the subject with nucleic acids encoding the A1-A2 domain of Factor V.

10. The method of claim 1, the method further comprising obtaining or having obtained a biological sample from the subject.

11. The method of claim 1, wherein VTE comprises deep vein thrombosis (DVT) or pulmonary embolism (PE)

12. (canceled).

13. A method of identifying a subject undergoing anticoagulant treatment who is at risk of developing venous thromboembolism (VTE), the method comprising:
determining or having determined the presence or absence of Factor V Leiden (FVL) nucleic acid molecule in a biological sample obtained from the subject;
wherein:
when the subject has a nucleic acid molecule encoding FVL, then the subject has an increased risk of developing VTE; and
when the subject does not have a nucleic acid molecule encoding FVL, then the subject does not have an increased risk of developing VTE.

14. The method of claim 13, the method further comprising:
i) selectively depleting FVL in a subject that is heterozygous for a nucleic acid molecule encoding FVL; or
ii) replacing FVL in the genome of the subject that is homozygous for FVL with a functioning Factor V gene.

15. The method of claim 14, wherein selectively depleting FVL comprises administering to the subject an inhibitory nucleic acid molecule that hybridizes to an FVL nucleic acid molecule.

16. The method of claim 15, wherein the inhibitory nucleic acid molecule comprises an antisense nucleic acid molecule, a small interfering RNA (siRNA), and/or a short hairpin RNA (shRNA).

17. The method of claim 16, wherein the inhibitory nucleic acid molecule comprises an siRNA or an antisense nucleic acid molecule.

18. (canceled)

19. The method of claim 14, wherein selectively depleting FVL comprises administering to the subject an anti-FVL antibody, or antigen-binding fragment thereof.

20. The method of claim 14, wherein replacing FVL in the genome of the subject that is homozygous for FVL with a functioning Factor V gene comprises performing base-pair editing to replace the glutamine at position 506 of Factor V with an arginine.

21. The method of claim 14, wherein replacing FVL in the genome of the subject that is homozygous for FVL with a functioning Factor V gene comprises replacing the FVL gene in the genome of the subject with a Factor V gene.

22. The method of claim 14, wherein replacing FVL in the genome of the subject that is homozygous for FVL with a functioning Factor V gene comprises replacing the nucleic acids encoding the A1-A2 domain of the FVL in the genome of the subject with nucleic acids encoding the A1-A2 domain of Factor V.

23. The method of claim 13, the method further comprising obtaining or having obtained a biological sample from the subject.

24. The method of claim 13, wherein VTE comprises deep vein thrombosis (DVT) or pulmonary embolism (PE).

25. (canceled)
